(19) United States
(12) Patent Application Publication
(10) Pub. No.: US 2014/0064595 A1
(43) Pub. Date: Mar. 6, 2014

(54) APPARATUS AND METHOD FOR DETERMINING THE COAGULATION TIME OF BLOOD

(71) Applicants: Sascha Meyer Dos Santos, Frankfurt (DE); Sebastian Harder, Frankfurt (DE); Zeno V. Guttenberg, Muenchen (DE); Anita Zorn, Bruchsal (DE); Ute Klinkhardt, Frankfurt (DE)

(72) Inventors: Sascha Meyer Dos Santos, Frankfurt (DE); Sebastian Harder, Frankfurt (DE); Zeno V. Guttenberg, Muenchen (DE); Anita Zorn, Bruchsal (DE); Ute Klinkhardt, Frankfurt (DE)

(73) Assignee: Johann Wolfgang Goethe-Universitaet Frankfurt am Main, Frankfurt am Main (DE)

(21) Appl. No.: 14/111,032

(22) PCT Filed: Apr. 11, 2012
(86) PCT No.: PCT/EP2012/001575

§ 371 Date: Nov. 21, 2013

(30) Foreign Application Priority Data
Apr. 11, 2011 (DE) 10 2011 001 952.9

(51) Int. Cl. A61B 5/145

(52) U.S. Cl. CPC A61B 5/145
USPC 382/134

(57) ABSTRACT

An apparatus and a method for determining the coagulation time of blood are shown. Surface waves are injected into a blood sample (30) therein, which blood sample contains fluorescent microspheres (32). The fluorescence of the microspheres is excited, and the movements of the microspheres are optically monitored. The time of coagulation can be determined using the deceleration or the standstill of the movement of the microspheres.

### FIELD OF THE INVENTION

The present invention lies in the field of medical engineering. More specifically, the invention relates to an apparatus and a method for determining the coagulation time of blood, use thereof, and an associated sample vessel.

### RELATED PRIOR ART

The coagulation or clotting of blood is a necessary process for stopping internal and external bleeding. In the case of coagulation under physiological conditions, two sub-processes are normally involved. One sub-process is based on thrombocytes, which normally initiate the clotting cascade. If a blood vessel is damaged, the thrombocytes typically adhere to the vessel opening, stick to one another and thus produce the initial wound closure. The second process concerns what is known as plasma clotting, in which the closure, which is still loose, is reinforced by the formation of fibrin threads in order to form a blood clot, also referred to as a thrombus.

Although the ability to coagulate is a vital property for limiting blood loss in the event of injury, it also poses risks for humans. The best-known example for this is what is known as thrombosis, in which a thrombus forms in a vessel and blocks it. Thromboses are often produced in veins, specifically the deep leg veins. If the thrombus detaches, it may reach the pulmonary artery, for example via the inferior vena cava and the right atrium of the heart, and may block said artery, which may lead to a pulmonary embolism. A frequent cause of thromboses is an illness-related rise in the coagulation ability of the blood, which can be counteracted using coagulation-inhibiting drugs, for example Heparin or Argatroban. For diagnosis of the increased tendency to coagulation or in order to set the correct dose of the coagulation inhibitor, the coagulation ability of the blood must be tested, for example by measuring the coagulation time of a blood sample in an apparatus suitable for this purpose. The correct dosage of coagulation-inhibiting agents is extremely critical since an excessive lowering of the tendency to coagulation in turn entails the risk of uncontrolled bleeding, even in the case of relatively minor injuries.

A particularly reliable and precise monitoring of the coagulation ability of the blood is also necessary prior to surgical interventions, because it must be ensured that the bleeding of wounds produced unavoidably during the process will also stop reliably. Another important application is emergency medicine. If, for example in the event of an injury, internal bleeding is suspected, it is extremely important to determine whether the blood is sufficiently capable of coagulation. For example, the injured person for unknown reasons may regularly take coagulation-inhibiting drugs, which would then cause increased internal bleeding. For the emergency doctor, it would therefore be important to be able to test quickly and reliably the ability of the blood to coagulate as a routine measure in order to be able to initiate suitable countermeasures where necessary.

A large number of apparatuses and methods for measuring the clotting ability of blood are known from the prior art. These include a large number of mechanical apparatuses, for example as described in EP 0 596 222 A1, in which blood clotting is measured in a measuring cell on an enclosed ball. Other mechanical methods use a test cell with a capillary path through which blood is pumped back and forth. The flow rate is measured and in turn indicates the degree of clotting of the blood, for example see U.S. Pat. No. 5 372 946. In other methods, coagulation is measured optically, for example based on the light permeability of a sample in a capillary, as is described for example in WO 89/06803.

DE 10 2008 026 009 B4 describes a method for determining a viscosity with use of acoustoelectric resonators. The viscoelastic medium is applied as a measurement medium to an acoustoelectric resonator, which, besides a most strongly pronounced resonance field, has further secondary modes that neighbour one another closely in terms of the frequency. One-port resonators based on surface acoustic waves (SAWs) can be used as acoustoelectric resonators. The admittance curve on the acoustoelectric resonator is measured in accordance with the frequency in a frequency range of +/−10% of the frequency of the most strongly pronounced maximum of the admittance value. In an iterative method, the viscosity of the medium can be concluded from this measurement. This document, inter alia, proposes applying the method for characterisation of dynamic processes, such as the determination of the clotting behaviour of blood.

### SUMMARY OF THE INVENTION

Although a large number of devices for determining the coagulation time of blood are known, there is still a need for improvements. An advantageous apparatus combines a number of properties, including good reproducibility of the measured results, simple operation, a quick analysis time, and a reasonable equipment cost. The object of the invention is to provide an apparatus and a method having such properties.

An apparatus for determining the coagulation time of blood which provides advantages in all these aspects is defined in claim 1. A corresponding method and use are defined in claims 13 and 17 respectively. Claim 10 defines a sample vessel for use with the apparatus according to the invention and the method according to the invention. Advantageous developments are specified in the dependent claims.

In accordance with the invention, an apparatus for determining the coagulation time of blood is provided, comprising:
a sample vessel for receiving a blood sample, or a receptacle for a sample vessel,
a device for generating surface waves, which are suitable for mixing a blood sample in the sample vessel,
a light source, which is suitable for exciting fluorescence of microspheres contained in the sample vessel, and
means for monitoring the movement of the fluorescent microspheres and for determining the coagulation time on the basis of the deceleration or standstill of this movement.

It should be noted that in this context the term “blood sample” is understood to mean any liquid in which blood is contained, but that further components may also be present, in particular calcium chloride for recalcification of the blood, and possibly a coagulation simulator, such as kaolin. Although whole blood is preferably examined within the scope of the invention, some components of the blood may optimally also be removed in the sample. Furthermore, it is shall be understood that the sample vessel as such does not necessarily have to be a component of the protected apparatus, but in contrast can be sold for example as a disposable article independent of the apparatus as such. In this case, the apparatus has a receptacle however, that is to say any type of mounting surface or holder for such a sample vessel suitable for use with the apparatus.

The apparatus according to the invention therefore makes it possible to mix the blood sample by means of surface waves that are coupled into the blood sample. As long as the blood does not coagulate, it is kept in movement by the surface waves and this movement is made visible (although not with the naked eye) by the fluorescent microspheres contained in the blood sample and can hence be optically monitored. As soon as coagulation initiates, the fluorescent microspheres will strongly decelerate however or come to a standstill, which can likewise be observed, such that the moment of coagulation can be determined.

Of course, coagulation as such is a process, and therefore there is not strictly speaking a clearly defined “moment” of coagulation. Instead, criteria are defined within the scope of the invention, on the basis of which the “moment of coagulation” is established. Provided these criteria are applied consistently, a reproducibility of the measured values of the coagulation time is provided that is found remarkable by the inventors. These measured values can then be associated by means of comparison or reference measurements with the clinically relevant dose range in anticoagulation therapy.

In an advantageous embodiment, the means for monitoring the fluorescent microspheres comprise an imaging optics that is suitable for imaging an image of the fluorescent microspheres onto an image sensor, and an image analysis device which is suitable for ascertaining the extent of the movement of the fluorescent microspheres in a blood sample on the basis of images recorded in succession by the image sensor, and for ascertaining the moment at which the extent of the movement falls below a predetermined threshold value.

In an advantageous embodiment, the image analysis unit is designed to ascertain a similarity or correlation between two images recorded chronologically one after the other and to determine the extent of the movement on the basis of this similarity or correlation.

In accordance with this embodiment, the extent of the movement is therefore quantified by the similarity between chronologically successive images. It is clear that two images recorded in a specific interval will be less similar, the more heavily pronounced the current movement is, and that the similarity apart from fluctuations in the measuring equipment will be perfect if the blood with the microspheres contained therein comes to a standstill. In this regard, an analysis of the correlation or of a similarity of chronologically successive images is a suitable, quantifiable measure, on the basis of which the movement can be monitored and a deceleration or standstill of the movement can be detected.

A further advantage of this embodiment is that suitable image analysis programs that ascertain and quantify a similarity or correlation between images are already known from other applications and can be adopted for the purposes of the invention.

The device for generating surface waves preferably comprises a piezoelectric substrate, on which an electrode structure is formed, and an alternating current source, which is connected or is connectable to the electrode structure. The piezoelectric substrate consists for example of lithium niobate (LiNbO3).

The electrode structure preferably comprises at least two comb-like electrode arrangements each with a plurality of parallel fingers that are arranged so as to engage with one another at least in part. When an alternating current signal is applied to the two comb-like electrode arrangements, an electric field is generated between the adjacent electrode fingers and the piezoelectric effect leads to a deflection, corresponding to the excitation voltage, of the piezoelectric material and, with suitable frequency of the alternating current signal, to the generation of surface waves.

A particular advantage of the apparatus according to the invention is that it can be highly miniaturised. This is true for the equipment set-up itself, which can be kept extremely small and compact and is therefore ideally suitable for a portable device which can also be used outside medical laboratories, for example directly at the hospital bedside, in care establishments, in emergency vehicles or in a patient's domestic environment. A further aspect of miniaturisation concerns the necessary quantity of the blood sample. It has been found that the method according to the invention can be carried out very successfully with use of blood samples that contain merely 5 μl of whole blood, that is to say one drop of blood. The sample vessel therefore preferably has a receiving volume of less than 40 μl, preferably less than 20 μl. The ability of the apparatus according to the invention to generate meaningful and reproducible results with very small blood sample volumes is advantageous in particular in the case of animal tests carried out on small animals, such as mice, which only have a small quantity of blood.

In an advantageous embodiment, the sample vessel consists of a biocompatible polymer material, wherein polydimethylsiloxane has proven in particular to be advantageous.

In accordance with a further aspect, the invention comprises a sample vessel for use in an apparatus according to one of the above-described embodiments, the sample vessel being prefilled with fluorescent microspheres and having an orifice for addition of blood. This sample vessel may in particular be a disposable product. The term “pre-filled” indicates that the sample vessel is already pre-filled with the microspheres at the factory and can be stored by the user in this pre-filled state. Only at the moment of the actual determination of the coagulation time is the blood then added. In addition, the sample vessel may also be pre-filled with calcium chloride as recalcification reagent and, where applicable, a coagulation stimulator, such as kaolin.

In a particularly preferred embodiment, the sample vessel pre-filled with microspheres is pre-treated to exhibit a negative pressure, such that it can draw in the suitable quantity of blood during its use without the need for complex pipetting or the like. This is of significance in particular for applications outside laboratory conditions, for example in emergency vehicles, care establishments or at home. This is also of great advantage in a hospital environment however, for example if the test is carried out directly at the patient's bedside, and the result is therefore immediately available.

A further possibility for a quasi-automatic filling of blood into the sample vessel pre-filled with microspheres lies in forming the geometry and/or inner surface of the container such that a blood sample is drawn into the sample vessel merely by capillary action and without additional negative pressure produced at the time of filling. Such a filling process driven purely by capillary force is enabled due to the very low sample volumes required. Alternatively, the blood sample can be drawn into the sample vessel by microfluid techniques.

### BRIEF DESCRIPTION OF THE FIGURES

Further advantages and features of the invention will become apparent from the following description, in which the invention will be described on the basis of an exemplary embodiment with reference to the accompanying drawings, in which:

FIG. 1 shows a schematic set-up of an apparatus according to the invention,

FIGS. 2(a) to (e) show a sequence of schematic images that illustrate the functionality of the apparatus of the invention,

FIG. 3 shows the course over time of the correlation between chronologically successive images of blood samples from seven test persons not treated with drugs,

FIG. 4 shows the course over time of the coagulation between successive images of seven blood samples of a test person with different doses of Argatroban,

FIGS. 5(a) to (d) show bar graphs for coagulation times according to various doses of Argatroban for the method of the invention (FIG. 5(d)) and for three conventional test methods (FIGS. 5(a) to (c)),

FIG. 6 shows a graph illustrating the coagulation time according to a Heparin dose in five test persons, and

FIG. 7 shows a graph illustrating the coagulation time according to a dose of the thrombocyte-aggregation inhibitor Abciximab.

### DESCRIPTION OF THE PREFERRED EMBODIMENT

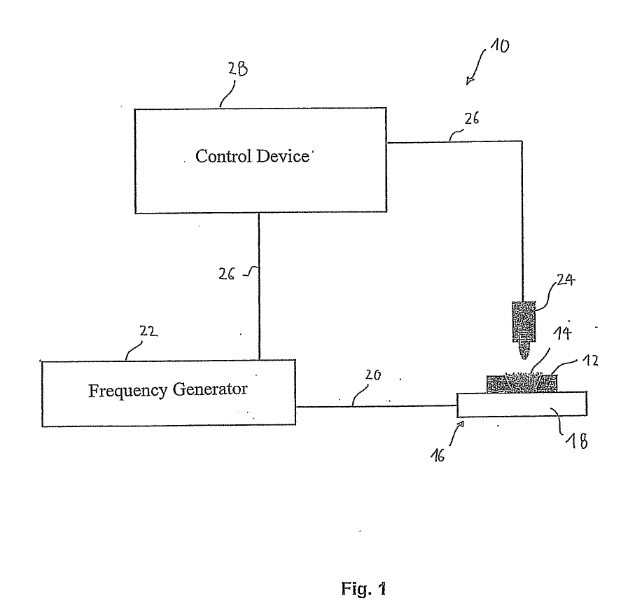

In FIG. 1, the set-up of an apparatus 10 for determining the coagulation time of a blood sample according to an embodiment of the invention is illustrated schematically. The apparatus 10 comprises a receptacle 12 for receiving a sample vessel 14. The receptacle is located on a device 16 for generating surface acoustic waves, which are also referred to in the literature as SAWs. The device 16 comprises a piezoelectric chip 18, on the upper face of which comb-like electrode arrangements (not shown) are formed, each having a plurality of parallel fingers that engage with one another. The electrode arrangements (not shown) are connected via a line 20 to an alternating current source, more specifically a frequency generator 22.

Above the sample vessel 14, a miniaturised fluorescence microscope 24 is arranged, which is known per se and does not need to be explained here in detail. The fluorescence microscope 24 comprises a light source (not shown) for exciting the fluorescence of microspheres (not shown in FIG. 1) contained in the sample vessel 14 and also comprises an imaging optics (not shown) which is suitable for imaging an image of the fluorescent microspheres onto an image sensor. Both the fluorescence microscope 24 and the frequency generator 22 are connected via data lines 26 to a control device 28.

Microspheres are known to a person skilled in the art. They are commercially available in various embodiments with the desired physiochemical properties, such as diameter, fluorescence wavelengths and surface chemistry. For further details, reference is made to the overview article “Microspheres for biomedical applications: preparation of reactive and labelled microspheres” by Reza Arshady, Biomaterials. 1993; 14(1):5-15 and “Polymer microbeads in immunology” by V\check{e}tvicka et al., Biomaterials. 1987 September; 8(5):341-5.

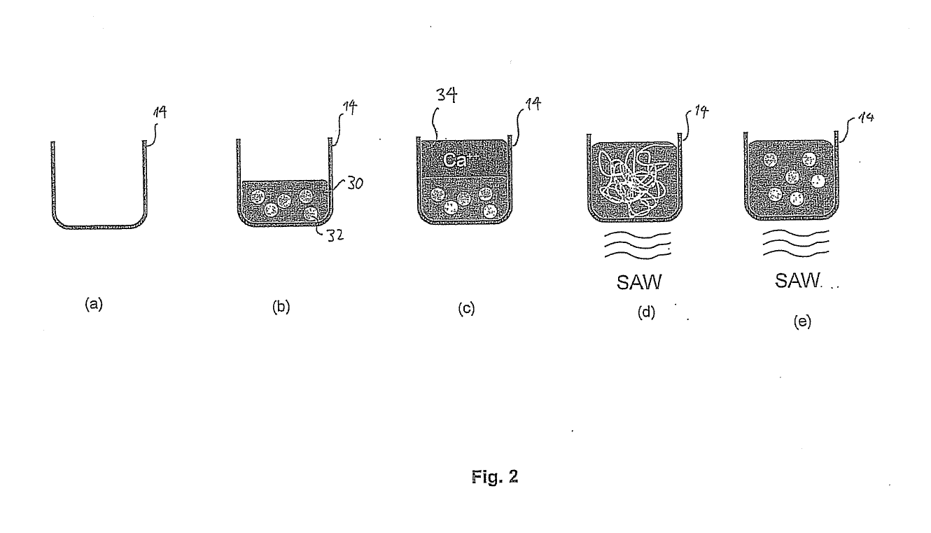

Next, the functionality of the apparatus 10 will be described with reference to FIG. 2.

FIG. 2(a) shows the empty sample vessel 14 in a schematic illustration.

FIG. 2(b) shows the sample vessel 14, which is filled with a blood sample 30 in which biocompatible fluorescent microspheres 32 are contained. Fluorescent microspheres 32 of this type are known from chemical and biological analytics. In the shown exemplary embodiment, they consist of a biocompatible material and have a diameter of approximately 1 μm.

As is shown in FIG. 2(c), calcium chloride is then added and forms Ca++ ions in the solution, which contribute to recalcification and thus initiate clotting.

With the aid of the device 16, surface acoustic waves are then generated and coupled into the sample liquid, as is illustrated schematically in FIG. 2(d). Graphically speaking, the surface acoustic waves coupled into the sample liquid act as a “nano-earthquake” and lead to intense mixing of the blood sample 30 and simultaneously to a rapid movement of the fluorescent microspheres 32, which is illustrated schematically in FIG. 2(d). This movement can be monitored with the aid of the fluorescence microscope 24 (see FIG. 1). To this end, besides an imaging optics, the fluorescence microscope 24 also contains an image sensor, recording digital images that are transmitted through the data line 26 to the control device 28.

When coagulation initiates, the mixing of the blood sample and therefore the movement of the microspheres 32 decelerates, in spite of ongoing excitation by the surface acoustic waves. This deceleration or standstill of the movement can be determined automatically by image analysis carried out by the control device 28.

In the preferred embodiment, the control device 28 carries out a correlation analysis of images of the fluorescence microscope 24 following one another in succession at regular intervals over time. It has been found that special software would not have to be developed for this correlation analysis, but that the use of a publicly available standard software, specifically the plug-in “CorrelationJ” of the software “ImageJ” has already delivered very good results.

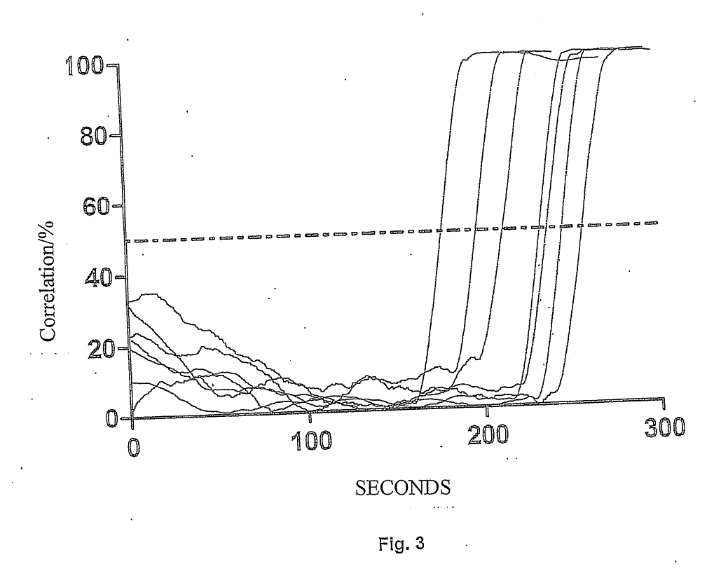

FIG. 3 shows the course over time of the correlation between successive images, this being provided with the set-up of FIG. 1 in blood samples from seven different test persons. The output parameter of the program, which represents the correlation, was standardised to 100%, corresponding to the case of maximum similarity or standstill of the sample.

As can be inferred from FIG. 3, all curves after an initial noise show a relatively rapid rise of the correlation, which corresponds to the coagulation process. In this embodiment, the moment at which the correlation curve reaches 50% was defined for each test person as the moment of coagulation. This limit value is large enough to prevent random noise being misinterpreted as coagulation. This limit value is in no way mandatory however, and for example a limit value of 80% could also be used to define a reproducible and reliable moment of coagulation. In this case, it is only important that the definition of the moment of coagulation is used consistently in order to ensure a reproducibility and a comparability between different measured values.

As can be inferred from FIG. 3, the coagulation times of the samples are spread in a region of approximately 80 s around a mean value of slightly more than 200 s. This spread is due to the fact that the coagulation capability of the blood and consequently the coagulation time measured using the apparatus of the invention fluctuates even in the case of healthy test persons.

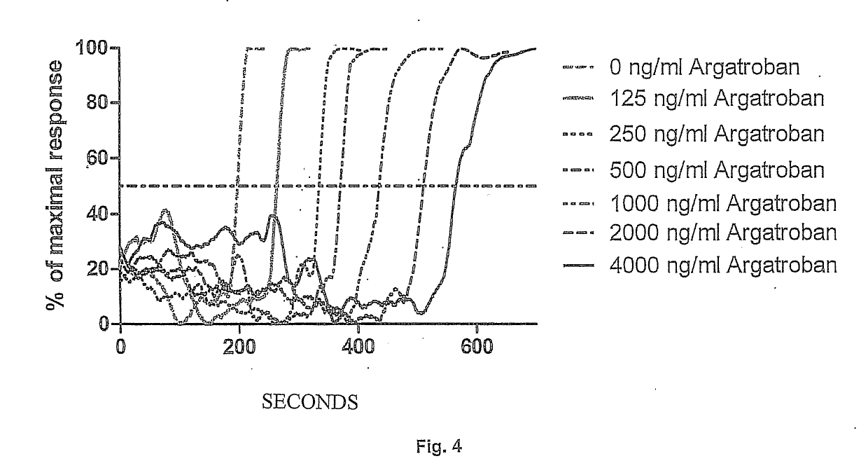

FIG. 4 likewise shows the course over time of the correlation, but for the same blood sample, to which different quantities of a coagulation-inhibiting agent, in this case Argatroban, have been added. As can be inferred from FIG. 4, the clotting-inhibiting action of Argatroban is visible directly by the extension of the coagulation time. The apparatus 10 of the invention thus makes it possible to test the action of a clotting inhibitor directly in whole blood. The dose can then be adapted in accordance with the result, for example.

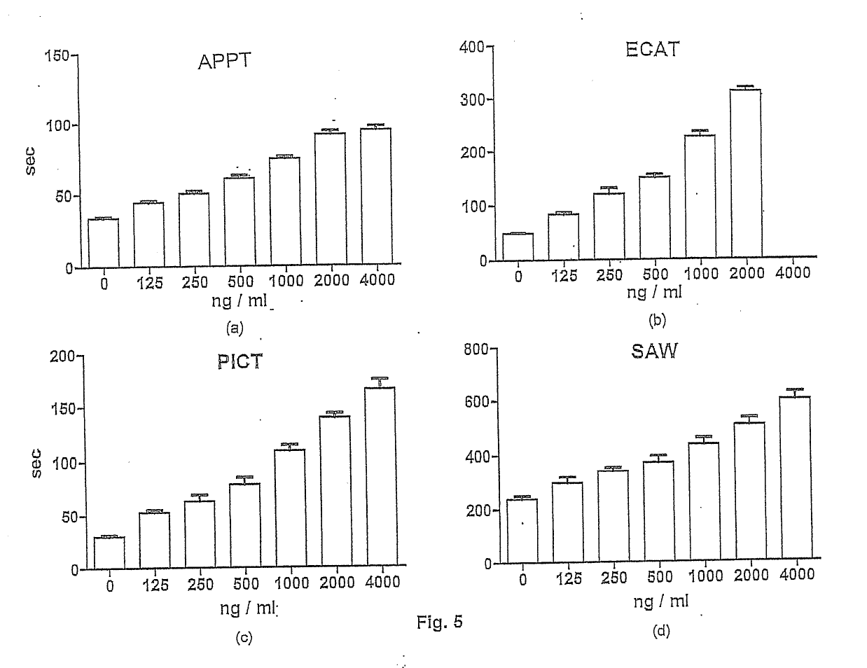

FIG. 5 shows comparison measurements for coagulation times that have been ascertained using different established methods (5(a) to (c)) and using the apparatus and the method of the invention (FIG. 5(d)). The APPT (FIG. 5(a)) measures the time required for thrombus formation after addition of phospholipids and clotting accelerators (such as kaolin, silica, or the like) in the recalcified plasma low in platelets. In the ECAT (Ecarin time) Essay (FIG. 5(b)), the meizothrombin formation is induced by the addition of the snake venom Ecarin and clotting is monitored by the change in the optical density in the sample after conversion of chromogenic substrates. In the PICT (prothrombinase induced coagulation time) Essay (FIG. 5(c)), the plasma sample low in platelets is mixed with defined quantities of factor Xa, phospholipids and the snake venom RVV-V. The clotting time is measured after recalcification.

FIG. 5 shows that the method of the invention delivers a characteristic monotonous correlation between coagulation time and drug dose, similarly to established, but much more complex methods that in principle can only be carried out by trained individuals, and therefore the method of the invention is just as suitable as the established methods for the determination of appropriate doses. It should be noted in particular that the ECAT method (FIG. 5(b)) can no longer be used for a dose of 4,000 mg/ml Argatroban, as a result of which the corresponding value has been omitted in the bar graph.

The particular advantage of the invention compared to these standard methods lies however in the fact that it can be implemented with extremely low equipment cost and is suitable in particular for a cost-effective portable unit that can be used readily outside a laboratory environment and does not require trained operators.

It should be noted that the coagulation times within the scope of the shown embodiment of the invention last slightly longer than with the established ATTP, ECAT and PICT methods, in which clotting accelerators, such as phospholipids, kaolin, etc., are used. Similar clotting accelerators can also be used however in the apparatus and the method of the invention, whereby the measurement times are likewise reduced.

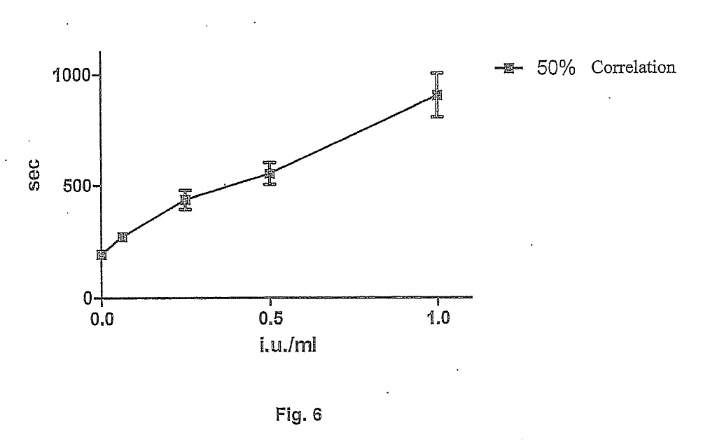

Lastly, the coagulation times for untreated blood and four different Heparin doses are shown in FIG. 6. The measurement is based on the values from five test persons. Here too, the characteristic dependence of the coagulation time on the dose can be seen, and therefore the apparatus and the method of the invention are ideal for setting the suitable dose.

The apparatus of the invention provides particular advantages outside a laboratory environment. A characterising feature of the apparatus and the method of the invention is that the blood does not need to be pre-treated in any way, but the whole blood provided can be easily used, of which even a quantity of just 5 μl is sufficient. In an advantageous embodiment, the appropriate dosing is already ensured by the manufacturer by means of the size of the sample vessel 14, which has been illustrated only schematically in FIGS. 1 and 2, and a pre-dosed supply of the recalcification reagent 34 (for example calcium chloride) plus optional coagulation accelerators. The apparatus of the invention can also be integrated however in existing units without difficulty, for example via a docking station.

The sample vessel 14 is preferably already pre-filled with a suitable quantity of fluorescent microspheres 32. Furthermore, the sample vessel 24 may already be pre-filled with the recalcification reagent 34 in a suitable dose. Alternatively, the recalcification reagent 34 could also be provided in pre-dosed containers (not shown). The user then only has to fill up completely the pre-filled sample vessel 14 with blood, place it in the receptacle 12 of the apparatus 10, and start the analysis program, for example by simply pressing a button. The control device 28 then outputs the coagulation time. Alternatively the control device 28 may of course also output other information associated with the measured coagulation time, for example a warning in the event of low coagulation capability, for example as would be advantageous in emergency medicine, or a specific dose proposal for a patient taking coagulation-inhibiting drugs in the long term and who can monitor his dose with the aid of the apparatus 10 in home use.

As can be seen from FIGS. 4 and 5, the efficacy of coagulation-inhibiting drugs, such as Argatroban and Heparin, can be evaluated quantitatively with use of the apparatus and the method of the invention. These drugs are agents that inhibit plasma coagulation. Further tests carried out by the inventors have shown however that the efficacy of thrombocyte-aggregation inhibitors can also be observed and measured by means of the method and the apparatus of the invention. Thrombocyte-aggregation inhibitors are used for example for surgical interventions on the heart and for short-term prophylaxis of myocardial infarction. A known example is Abciximab, which is a fab fragment of a monoclonal antibody, which binds the glycoprotein IIb/IIIa receptors on the surface of thrombocytes in an inhibitory manner and therefore blocks the binding sites for fibrinogens and other adhesion molecules.

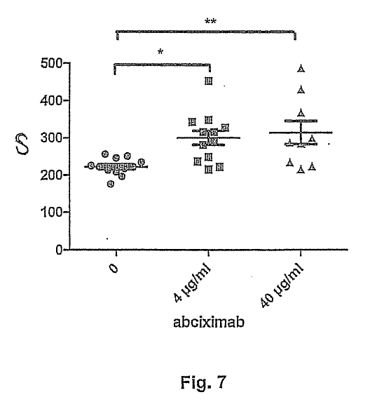

As mentioned in the introduction, the results presented here were obtained with the aid of a software for correlation analysis, said software not yet being optimised for the present application. As can be inferred from FIGS. 3 to 5, this image analysis is already sufficient to present the coagulation process in a very clearly identifiable manner in the correlation/time graph, from which a coagulation time could then in turn be ascertained. It should be mentioned however that the coagulation time is just one piece of information that can be obtained from the analysis of the images, but that the time evolution of the movement of the microspheres additionally provides further information concerning the hydrodynamic state of the blood sample, for example the time evolution of viscosity, etc. By providing specifically adapted image analysis programs and suitable SAW excitation patterns, it is possible to obtain further and more detailed information concerning the coagulation behaviour than just the coagulation time as such.

Whereas anticoagulants in the strict sense relate to substances that influence the clotting factors of blood plasma, coagulation can also be delayed by what are known as thrombocyte-aggregation inhibitors, which act on the blood platelets. An example for this is Abciximab, which is a fab fragment of a monoclonal antibody, which binds to the glycoprotein receptors on the surface of the blood platelet in an inhibitory manner. FIG. 7 shows measurement data of the moment of coagulation without addition of Abciximab, with an addition of 4 mg/ml, and with an addition of 40 mg/ml of Abciximab.

It can be inferred from FIG. 7 that the effect of the thrombocyte-aggregation inhibitor can be verified in the extension of the coagulation time. This is a surprising result in view of the simplicity of the equipment set-up, since the effect of thrombocyte-aggregation inhibitors can otherwise only be determined in complex methods with substantial equipment set-up.

### LIST OF REFERENCE SIGNS

10 apparatus for determining the coagulation time

12 receptacle for receiving a sample vessel 14

14 sample vessel

16 device for generating surface waves

18 piezoelectric chip

20 line

22 frequency generator

24 fluorescence microscope

26 data lines

28 control device

30 blood sample

32 microspheres

34 recalcification reagent

1-17. (canceled)

18. An apparatus for determining the coagulation time of blood, said apparatus comprising:
a sample vessel for receiving a blood sample, or a receptacle for a sample vessel,
a device for generating surface waves suitable for mixing a blood sample in the sample vessel,
a light source suitable for exciting the fluorescence of fluorescent microspheres contained in the sample vessel, and
a monitoring device for monitoring the movement of the fluorescent microspheres and for determining the coagulation time on the basis of the deceleration or the standstill of the movement.

19. The apparatus according to claim 18, in which the monitoring device comprises:
an imaging optics suitable for imaging an image of the fluorescent microspheres if contained in a blood sample, onto an image sensor, and
an image analysis device suitable for establishing the extent of the movement of the fluorescent microspheres in a blood sample on the basis of images recorded in succession by the image sensor, and for establishing the moment at which the extent of the movement falls below a predetermined threshold value.

20. The apparatus according to claim 19, wherein the image analysis unit is designed to establish a similarity or correlation between two images recorded chronologically one after the other and to establish the extent of the movement on the basis of this similarity or correlation.

21. The apparatus according to claim 18, wherein the device for generating surface waves comprises a piezoelectric substrate on which an electrode structure is formed,
and comprises an alternating current source, which is connected or is connectable to the electrode structure.

22. The apparatus according to claim 21, wherein the piezoelectric substrate consists of lithium niobate (LiNbO3).

23. The apparatus according to claim 21, wherein the electrode structure comprises at least two comb-like electrode arrangements each having a plurality of parallel fingers which are arranged so as to engage with one another at least in part.

24. The apparatus according to claim 1, wherein the sample vessel has a receiving volume of less than 40 μl.

25. The apparatus of claim 24, wherein the sample vessel has a receiving volume of less than 20 μl.

26. The apparatus according to claim 18, wherein the sample vessel consists of a biocompatible polymer material, including polydimethylsiloxane.

27. The apparatus according to claim 18, wherein the apparatus is portable and battery-operated.

28. A sample vessel for use with an apparatus for determining the coagulation time of blood,
said apparatus comprising:
a sample vessel for receiving a blood sample, or a receptacle for a sample vessel,
a device for generating surface waves suitable for mixing a blood sample in the sample vessel,
a light source suitable for exciting the fluorescence of fluorescent microspheres contained in the sample vessel, and
a monitoring device for monitoring the movement of the fluorescent microspheres and for determining the coagulation time on the basis of the deceleration or the standstill of the movement,
which sample vessel is pre-filled with fluorescent microspheres and has an orifice for the addition of blood.

29. The sample vessel according to claim 28, which is pre-filled with a recalcification reagent, and which contains a coagulation stimulator.

30. The sample vessel according to claim 28, which is formed as a disposable product and/or is pretreated such as to exhibit a negative pressure or, due to its geometry and/or inner surface nature, is designed to receive a blood sample by means of capillary action and without additional negative pressure generated at the moment of filling.

31. A method for measuring the coagulation time of blood, said method comprising the following steps:
generating surface waves and coupling the surface waves into a blood sample that contains fluorescent microspheres,
exciting the fluorescence of the microspheres,
optically monitoring the movement of the microspheres and determining the moment of coagulation on the basis of the deceleration or standstill of the movement.

32. The method according to claim 31, wherein images of the blood sample with fluorescent microspheres are recorded and the deceleration or the standstill of the microspheres is ascertained on the basis of the similarity or the correlation between successive images.

33. The method according to claim 31, wherein the blood sample contains less than 20 μl, of blood.

34. The method according to claim 31, wherein the blood sample comprises calcium chloride for recalcification.

35. The method of claim 34, wherein the blood sample further comprises a coagulation stimulator.

36. The method of claim 31, where the method is carried out outside a medical laboratory environment.

37. The method of claim 36, where the method is carried out at a hospital bedside, in an emergency vehicle, in a care establishment, or in a home environment.
